(19) United States
(12) Patent Application Publication
(10) Pub. No.: US 2026/0240426 A1
(43) Pub. Date: Aug. 20, 2026

(54) SYSTEM AND METHOD FOR DRYING AND STORAGE OF ENDOSCOPES

(71) Applicant: Medivators Inc., Minneapolis, MN (US)

(72) Inventor: Timothy W. Herbst, Prior Lake, MN (US)

(21) Appl. No.: 19/455,695

(22) Filed: Jan. 21, 2026

Related U.S. Application Data
(60) Provisional application No. 63/760,217, filed on Feb. 19, 2025.

(51) Int. Cl. A61B 1/12

(52) U.S. Cl. CPC A61B 1/121

(57) ABSTRACT

Various embodiments disclosed relate to methods and systems for drying endoscopes, including a nitrogen-based drying pack. A system may include a nitrogen source to provide nitrogen gas. A system may include a drying pack fluidly connected to the nitrogen source, the drying pack comprising: a hermetically sealable pouch shaped to receive an endoscope, an inlet block fluidly connected to the nitrogen source and having one or more endoscope connections, and an outlet block configured to exhaust the hermetically sealable pouch. A system may include a gas control system coupled to the drying pack, with the gas control system adapted to adjust flow of the nitrogen gas into the drying pack.

### PRIORITY CLAIM

This application claims the benefit of priority to U.S. Provisional Patent Application No. 63/760,217, filed Feb. 19, 2025, and titled “SYSTEM AND METHOD FOR DRYING AND STORAGE OF ENDOSCOPES”, which is incorporated herein by reference in its entirety.

### BACKGROUND

Endoscopes are used in a variety of medical procedures. Often, after such procedures, the endoscopes are reprocessed through machines such as automated endoscope reprocessors (AERs) that clean and disinfect the endoscopes. After such reprocessing, endoscopes are dried through a variety of methods, such as through drying cabinets and other devices.

### SUMMARY OF THE DISCLOSURE

In some aspects, the techniques described herein relate to a system including: a nitrogen source to provide nitrogen gas; a drying pack fluidly connected to the nitrogen source, the drying pack including: a hermetically sealable pouch shaped to receive an endoscope; an inlet block having one or more endoscope connections, the inlet block fluidly connected to the nitrogen source; an outlet block configured to exhaust the hermetically sealable pouch; a gas control system coupled to the drying pack, the gas control system adapted to adjust flow of the nitrogen gas into the drying pack.

In some aspects, the techniques described herein relate to use of a drying pack for drying an endoscope, the pack including: a pouch sized to accommodate a coiled endoscope on a support tray provided therein, with the pouch being sealable along one side; an endoscope connection manifold bonded to an interior of the pouch that fluidly connects respective hook-up lines to one or more internal channels of an endoscope placed in the pouch; an inlet valve configured to receive a fluid, the inlet valve fluidly connected to respective hook-up lines via the manifold; and an exhaust valve fluidly connectable to the interior of the pouch, wherein, when the pouch is connected to a fluid source, the fluid is configured to enter the pouch through the inlet valve, flow through manifold and the respective hook-up lines, flow through the one or more internal channels of the endoscope, exit the endoscope at a distal tip of the endoscope, flow inside the pouch in a circulatory manner around an exterior of the endoscope, and exit the pouch through the exhaust valve.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 depicts a system including a nitrogen gas (N₂) pack in an example.

FIG. 2 depicts a schematic of a nitrogen gas (N₂) pack in an example.

FIG. 3 depicts use of the nitrogen gas (N₂) pack with an endoscope in an example.

FIG. 4A depicts a flow chart of a method of using the nitrogen (N₂) pack in an example.

FIG. 4B depicts a flow chart of a method of using the nitrogen (N₂) pack in an example.

FIG. 5 depicts an example computer device on which the methods herein can be executed in an example.

### DETAILED DESCRIPTION

Discussed herein is a system for drying and storing endoscopes using ultra-high purity nitrogen gas (N₂). The system includes a packaging unit—also referred to herein as a “pack”—that can be used to store and dry an endoscope. The pack incorporates a nitrogen flow system. In use, ultra-high purity nitrogen gas is fed into the pack, simultaneously drying both the internal channels and the exterior surfaces of the endoscope. This process creates an inert, dry environment that inhibits microbial growth.

Mechanically, the system includes a hermetically sealable pouch with an inlet block and outlet block, with each block heat sealed to the pouch. In some examples, a baffle is located in the middle of the pouch around which an endoscope can be wound, and a rigid tray is included inside the pouch to support the endoscope when placed in the pouch. The channels of the endoscope can be hooked up to an endoscope connection manifold or connection unit provided at the inlet block, through which the ultra-high purity nitrogen can be flowed into the respective endoscope channels via respective hook-up lines. The nitrogen can flow out of the endoscope channels at their terminal ends (e.g., at the distal tip of the endoscope) and be expelled to flow within the pouch, around the external surfaces of the endoscope, in a cyclonic flow. The fluid can be exhausted out of the pouch via the outlet block.

The system can provide several advantages by offering more efficient drying, reduced risk of contamination, and potentially shorter processing times. It eliminates the need for chemical disinfectants and vacuum pulling, while also reducing the chance of operator error compared to manual drying methods.

More generally, an atmosphere of nitrogen can reduce and prevent colonies of aerobic microbials (which cannot survive without oxygen), and common gut anaerobes like Bifidobacterium. Such types of anaerobes may need a minimum of trace carbon dioxide to function, and thus, a high concentration of nitrogen can create an environment that is not supportive of most types and species of bacteria. For these reasons, nitrogen has been used in biopharma, food packaging, and semiconductor industries for packaging, manufacturing, and prevention of oxidation.

The following system includes the use of ultra-high purity nitrogen. As used herein, “ultra-high purity” nitrogen, also referred to as “ultra-pure carrier” nitrogen, is defined as nitrogen gas of at least 99.9993% purity, grade N5.3 or greater, with no more than 1 ppm of oxygen or water residuals, no more than 1 ppm combined carbon dioxide and carbon monoxide residuals, and no more than 0.5 ppm total vaporous hydrocarbon residuals. It will be understood that other nitrogen gas mixtures of a higher purity, such as of at least 99.9999% purity, grade N6.0, can also be used with the present approaches. Further, the present pack or pouch can also be used with other gas mixtures and concentrations that inhibit microbial life.

Thus, a closed system for processing one or more endoscopes using such ultra-high purity nitrogen could provide benefits. The systems and methods discussed herein leverage an efficient use of space and circulation style blow-down purging to achieve efficient drying in a closed environment with ultra-high purity nitrogen. In addition, the proposed system can dry endoscope lumens and exterior surfaces at the same time with a reduced chance of operator missteps. This also reduces the need for additional components such as wipes and air guns, reducing total use of resources and minimizing process time, while still achieving acceptable levels of dryness.

The use of an ultra-high purity nitrogen blow-down technique with circulatory flow to simultaneously dry the lumens and exterior surfaces of a reprocessed endoscope can be aided by the geometry of a specifically designed aseptic package that is subsequently used for storage and that is readily transportable. Use of ultra-high purity nitrogen to purge and dry lumens in an automated endoscope reprocessor (AER) cycle can also offer an improvement in drying performance, such as if the final purge and dry cycles were extended. The proposed pack herein can provide an immediate drying benefit that can be realized on products dedicated to drying.

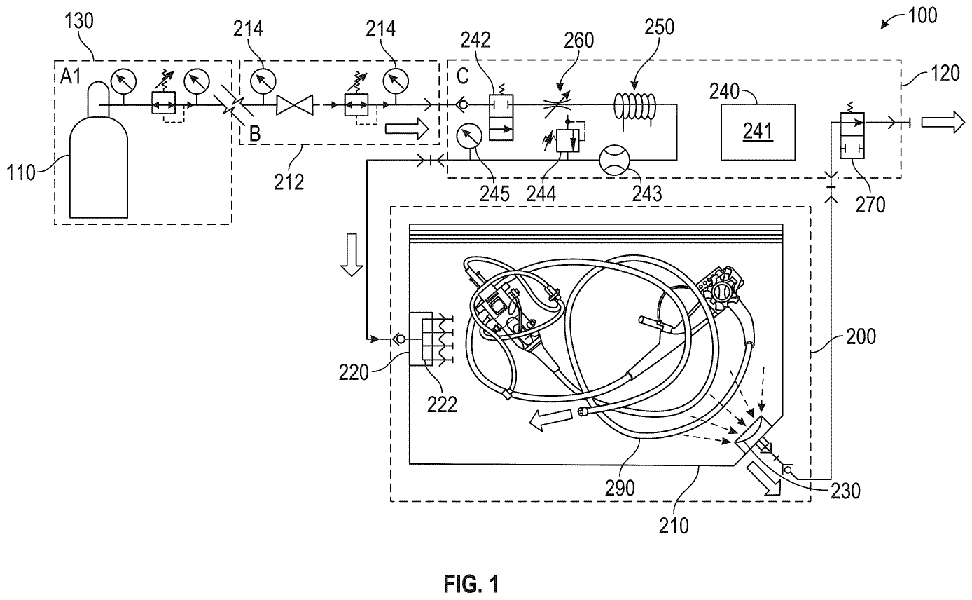

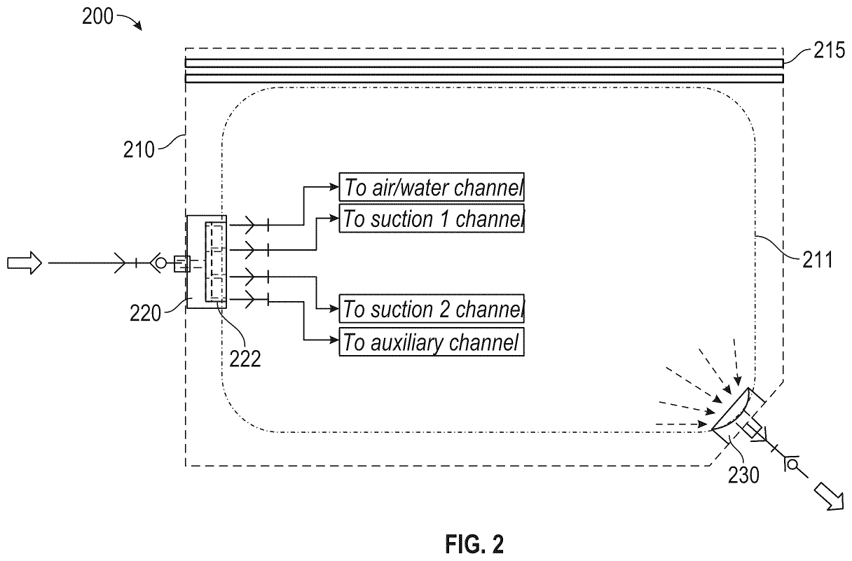

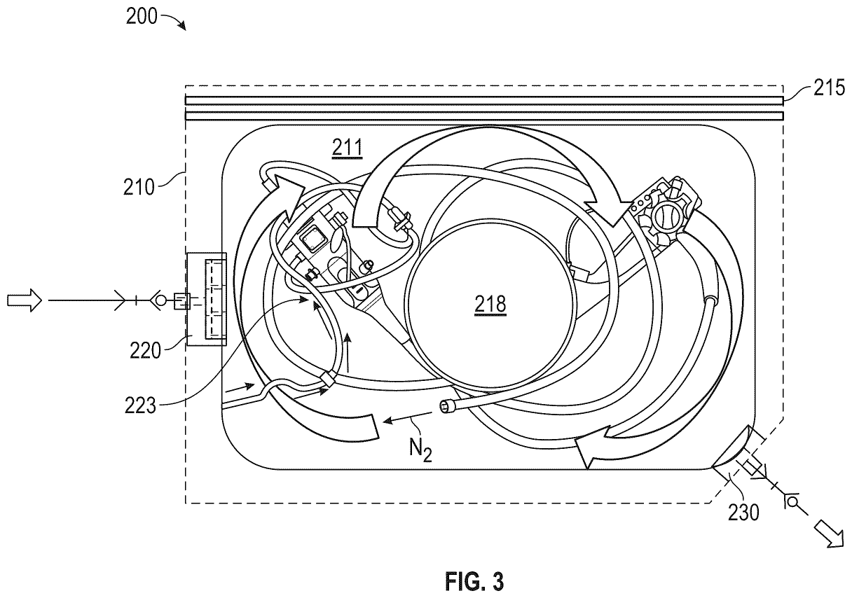

FIG. 1 depicts a system 100 including an N₂ pack 200 in an example, while FIG. 2 depicts a broken out view of the N₂ pack 200 in an example with no endoscope present. FIG. 3 depicts use of an endoscope in an alternative configuration of an N₂ pack 200 having a baffle 218 and tray 211 integrated therein. In each of these examples, the N₂ pack 200 can be fitted with a push-to-connect port with integral shut-off or check valve to accept a flow stream of ultra-high purity nitrogen at ambient or slightly elevated (45°-55° C.) temperature. FIGS. 1, 2, and 3 will be discussed together.

The system 100 can include a nitrogen source 110, the pack 200, a gas control system 120, and a storage location 130. The pack 200 can include a hermetically sealable pouch 210, an inlet block 220 with endoscope connections 222, and an outlet block 230. The system 100 can further include a nitrogen gas pressure-reducing regulator 212 with gauges 214, a system-integrated output device 240 with user interface 241, and a temperature regulation element 250.

The nitrogen source 110 is located at the storage location 130 and can be fluidly connected to the gas control system 120 and the pack 200. The storage location 130 can be configured to store ultra-high purity nitrogen. For example, the storage location 130 can be a tank or other pressurized container suitable for storage of nitrogen gas. The source of ultra-high purity (e.g., 99.9993% pure, grade N5.3) nitrogen can include a refillable cylinder that is located and managed according to appropriate safety standards for medical facilities such as National Fire Protection Association (NFPA) standards, e.g., NFPA-99. As explained above, such ultra-high purity/N5.3-grade Nitrogen (99.9993% pure) translates to less than 1 ppm O₂, less than 1 ppm H₂O, less than 1 ppm combined CO₂ and CO, and less than 0.5 ppm total vaporous hydrocarbons (THC) (e.g., “oils”).

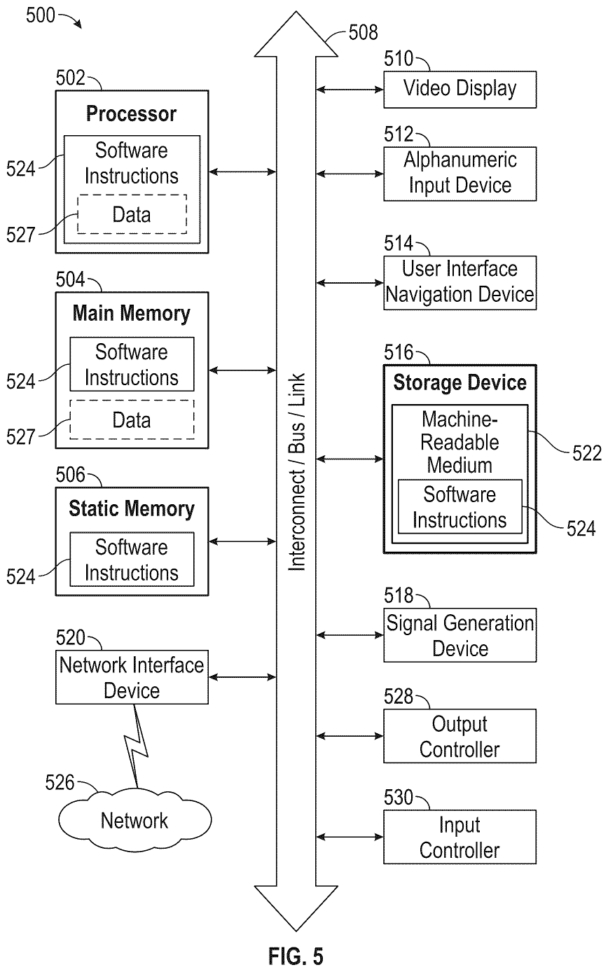

The gas control system 120 can include a controller or other control module (not depicted in FIG. 1, but optionally implemented by the computer device of FIG. 5 or a similar control system), having circuitry and programmed logic that is adapted to automatically adjust flow and/or pressure of the nitrogen gas from the nitrogen source 110 in the storage location 130 to the pack 200. The gas control system 120 can include and actuate one or more valves such as to regulate the flow and/or pressure of nitrogen into the system 100. The dedicated gas control system 120 can support the pack 200 and cycle two-way control valves to establish the dry cycle duration.

The gas control system 120 can provide a regulated supply of pressurized ultra-high purity N₂ gas from a local or remote source, and can include or use the nitrogen gas pressure-reducing regulator 212 and gauges 214 to regulate (e.g., reduce or increase) and monitor the flow of the nitrogen gas from the nitrogen source 110. In an example, the regulator 212 and other components of the gas control system 120 may be actuated by automatic controls or valves.

The gas control system 120 can control the pressure and flow of the N₂ gas with the use of various flow control and pressure valves and sensors. First, a two-way gas inlet valve 242 (toggled between on/off states) is shown to control the intake of the N₂ gas from the regulated supply. Next, a flow control needle valve 260 is used to control the flow of the N₂ gas. The gas control system 120 can adjust the flow of N₂ gas via an operator manually actuating the flow control needle valve 260, or by automatic actuation directed by a controller (e.g., adjusting a suitable electrically-actuated automated needle valve).

The gas control system 120 can additionally include a temperature regulation element 250. The temperature regulation element 250 can be configured to maintain the temperature of the N₂ gas entering the pack 200 in a range from about 45° C. to about 55° C. The temperature regulation element 250 can be a resistance coil or an infrared heater in an example.

The gas control system 120 can also include a mass flow meter 243 for monitoring the flow of the N₂ gas. The mass flow meter 243 may provide a flow readout on the meter itself or as an analog/digital input to a controller. The amount of N₂ gas flow may be manually or automatically controlled in the gas control system 120 to ensure efficient gas use and drying process time, depending on endoscope model.

The use of the flow control needle valve 260 and the mass flow meter 243 can be coordinated to fine tune the flow of pressurized N₂ gas based on the actual delivery pressure and temperature into the pouch. The flow rate may need to be varied depending on size and complexity of the endoscope in the pouch, which can be adjusted based on the readings of the mass flow meter 243. For example, an operator could manually adjust the flow control needle valve 260 (e.g., via a knob on a valve control module) or a controller could automatically adjust an automated needle valve based on the mass flow meter input and according to a scaled target range for mass flow of N₂. For instance, a N₂ mass flow rate readout can be captured by the mass flow meter 243 after some initial time period (e.g., 15 seconds), with reference to a scaled target range of mass flow rate and any user-selected previous input to the controller (e.g., that defined the size and complexity of the endoscope to be dried).

A pressure relief valve and pressure sensor can be used to prevent accidental excessive pressure from being applied into the pack 200. The gas control system 120 can include a pressure relief valve 244, using a mechanical valve that provides pressure relief for some fixed amount (e.g., in excess of 5 pounds per square inch gauge (psig)). The gas control system 120 can include a pouch pressure sensor 245 to provide analog/digital input of a pressure reading to a controller. For example, the pressure reading can provide an informative measurement to be output in the user interface 241.

The pack 200 can provide a hermetically sealable pouch 210 for drying an endoscope that includes residual liquids from cleaning and high-level disinfection reprocessing. FIG. 1 depicts the pack 200 having an endoscope 290 placed in the pouch 210, whereas FIG. 2 depicts the pack 200 providing the pouch 210 in an empty state. FIGS. 1 and 2 depict the pack 200 as including the pouch 210, an inlet block 220 providing endoscope connections 222 from a manifold, and the outlet block 230. FIG. 3 likewise depicts the pack 200 as including the pouch 210, an endoscope support tray 211, the baffle 218, the inlet block 220, and the outlet block 230. The pack 200 can be operably connected to the gas control system 120, with use of the system-integrated output device 240, the user interface 241, and the temperature regulation element 250.

The pack 200 can be optimized to minimize wasted space while still allowing easy ingress and egress of a properly coiled endoscope 290 (e.g., a colonoscope with less than ~20″ bend radius) to increase velocity around the exterior of the endoscope and inner walls of the pack 200. In some cases, the interior of the pack 200 can be treated with a hydrophilic coating to promote faster evaporation from the surfaces. In some cases, the pack 200 can be made of USP Class VI polyethylene or polypropylene. In some cases, the pack 200 can be a rigid container.

The pack 200 can be fluidly connected to the nitrogen source 110. Nitrogen fluid from the nitrogen source 110 can be configured to flow in the inlet block 220 through at least one of the endoscope connections 222, through one or more internal channels of an endoscope 290, exit the endoscope 290, and proceed to flow around the exterior of the endoscope 290 within the hermetically sealable pouch 210, then exit through the outlet block 230.

The hermetically sealable pouch 210 can include or accept an endoscope support tray 211 sized and shaped for insertion and/or receipt of the endoscope 290. The hermetically sealable pouch 210 can include a resealable closing mechanism such as provided by a double track “press-to-seal” or “press-to-close” zipper 215 that is actuatable to seal the pouch 210. In other examples, a resealable gasketed clamp, a single-track zipper, a triple-track zipper, or other variations of a zipper or clamp mechanism may be used.

Accordingly, it will be understood that the pouch 210 can be maintained in a hermetically sealed state, preventing the exchange of gases between the inside and outside of the pouch, when the pouch is sealed and when the inlet and outlet are closed. After the dry cycle is complete, a small amount of nitrogen gas may remain in the pouch 210 to act as a preservation atmosphere for the enclosed endoscope. In this state, the hermetically sealable pouch 210 and the endoscope 290 included therein may be stored and transported.

FIG. 3 depicts a variation of the pack 200 and the pouch 210, including the baffle 218 positioned within a center area of the pouch interior. The baffle 218 can reside in the pouch 210 directly, or the tray 211 may include geometry to incorporate a center baffle. The baffle 218 can be constructed and shaped to allow for winding of the endoscope 290 therearound.

When nitrogen is moved within the pack 200, the fluid can flow in a cyclonic flow within the confines of the pouch 210, such as around the baffle 218. The presence of a baffle can prevent gas-streaming from inlet to outlet and thus ensure cyclonic flow action to promote drying of the endoscope exterior. Drying of the internal channels can be driven by the turbulent flow of dry heated ultra-high purity nitrogen to expel droplets and increase evaporation rate.

In another example, a bonded seal can be made around the inner ~8″ diameter of the pouch 210 to remove this wasted central space instead of using a baffle insert. In another example, use of a 90-degree sweep clamped seal that spans over two adjacent sides of the periphery of the pack could increase ease of access and also allow for replicating the near full-rounded corner geometry to the other three corners and further reduce wasted interior volume. In general, the fluid dynamics of circulatory flow could be benefited by use of a more rigid bounding container to permit the use of tighter clearances and/or possible use of contours or air-foils with stable geometry.

The inlet block 220 can be heat-bonded to the hermetically sealable pouch 210. The inlet block 220 can be configured to allow for fluid flow into the pack 200, such as into the endoscope 290 lumens or channels via the endoscope connections 222. The endoscope connections 222 can be coupled via one or more hook-up lines to an air/water channel, a first suction channel, a second suction channel, or other auxiliary channels of the endoscope 290. The endoscope connections 222 can be actuatable to fluidly connect the endoscope 290 to the nitrogen source 110.

The inlet block 220 can be contained within the hermetically sealable pouch 210 or can have a flexible tubing connector that extends outside the pouch 210, and the pouch can be heat-sealed around the outside diameter of this connector. The connector can incorporate an integral check valve that opens when its mating “insert” connector from an outside line is inserted. This outside line can come from the gas control system 120 that regulates and heats incoming nitrogen gas from the nitrogen source 110. For example, the integral check valve can be configured to automatically close when the fluid line is disconnected from the nitrogen source 110.

The inlet block 220 can serve as a manifold to distribute the incoming nitrogen gas from a single inlet connection to multiple branches (e.g., four branches, as shown in FIG. 2) to enable delivery to the respective internal channels of the endoscope 290. Inside the pouch 210, each branch connection to the inlet block can be made with a flexible tubing connector. Depending on the specific make and model of endoscope, a specific tube set can connect individual flexible tubes to these four connection points on the inlet block. The opposite ends of these tubes can have a specific connector that is adapted to fit a specific endoscope channel, most commonly: air/water, suction 1, suction 2, and auxiliary channels. A different tube set can be used depending on endoscope make and model.

The outlet block 230 can be configured to exhaust the hermetically sealable pouch 210. The outlet block 230 can be heat bonded to the hermetically sealable pouch 210. An exhaust valve can be located in the outlet block 230, fluidly connectable to the pouch interior. This exhaust valve may be fluidly connected to other valves, such as valve 270 (e.g., an on/off valve) in a controlled exhaust system.

A flow control needle valve 260 can be configured to actuate the inlet connection and the exhaust valve. The flow control needle valve 260 can be used to regulate the flow of incoming nitrogen gas to an acceptable range. The flow control needle valve 260 can indirectly affect exhaust flow by monitoring and controlling the resulting backpressure and temperature. The incoming flow should be high enough to achieve the target dry time and obtain a slight positive pressure in the pouch. Simultaneously, the flow should be low enough to prevent over-pressurizing the pouch and wasting ultra-high purity nitrogen. The outlet flow can be limited by a fixed-size orifice outlet valve with a slight negative pressure relative to ambient. The differential pressure inside the pouch can be slightly positive.

The system-integrated output device 240 can be configured to provide a user interface 241. The system-integrated output device 240 can be in communication with the gas control system 120. In an example, the user interface 241 can provide information for monitoring the system 100 and the pack 200. In another example, the user interface 241 can allow for input of control signals for the system 100. In another example, the user interface 241 can be a display that shows measurements, such as those taken by various digital gauges, or sensor signals communicated to an external computing device.

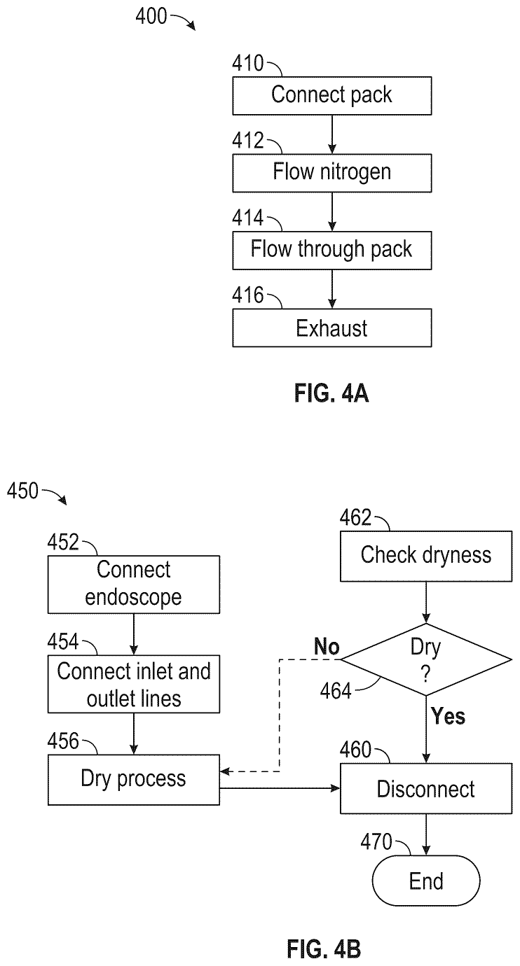

FIG. 4A depicts a flow chart of a method 400 of using the N₂ pack in an example, such as the pack 200 discussed above.

At block 410, a pack can be connected to a nitrogen source. Here, the pack can be fitted with a push-to-connect port with integral shut-off/check to accept a flow stream of ultra-high purity nitrogen at ambient or slightly elevated temperature. An endoscope can be connected internally in the pack. After making the hook-up connections, the endoscope can be gently coiled around a central baffle insert and loaded through a seam in the flexible walled pack along one side that would then be sealed with a clamp-style or a press-to-seal zipper style closure.

At block 412, nitrogen can be flowed into the pack and a connected endoscope. Inside the pack, a hook-up tubing header would route the N₂ flow into branches with terminal connections to the appropriate channels of an enclosed endoscope. A channel separator and vent cap can also be installed on the endoscope to enable unidirectional flow of N₂ out the nozzles at the distal tip into the confines of the sealed pack.

At block 414, flow can be pulled through the pack. Here, air can be pulled out the pack from the side that is located opposite the distal tip of the endoscope by an exhaust facility hook-up. The displaced air can be followed by extracting several pack volumes worth of fresh N₂ being fed in. In an example, about 0.24 SCFM of N₂ at 99.9993% purity can be used for about 10 minutes duration to fill and evacuate the pack twenty times over. Different cycles, times, and volumes can be used as desired, and tailored to specific packs and/or endoscopes.

At block 416, the nitrogen can be flown throughout the endoscope and pack. For example, as the N₂ flow exits the distal tip it can set up a circulatory flow pattern to increase fluid evaporation from exterior surfaces of the endoscope and the interior of the pack. In an example, after a desired time, the flow of fresh N₂ can be stopped (e.g., by a valve timer set by the user in a control module or other control) and the lines for N₂ entry and exhaust can be removed (such as through fittings featuring integral shut-offs), retaining an inert and dry blanket of nitrogen inside. In an example, the time can be ten minutes, or any other length suitable for such drying.

FIG. 4B depicts a flow chart of a method 450 of using a system such as system 100 above. At block 452, an endoscope can be inserted into the pack. The specific style of endoscope can be connected, such as through a specific type of tube set. The pack can then be sealed.

At block 454, a precondition flow of nitrogen can be done at programmed conditions. The inlet and outlet lines of the pack can be connected between the control module and the corresponding blocks on the pack. The desired dry time can be set, such as to 5 minutes or 10 minutes.

At block 456, the drying process can be performed. Optionally, at block 460, a level of dryness can be checked, such as with a reactive paper test strip (e.g., a copper sulfate test strip) inserted at a port of the pack 200. If not dry at block 462, the drying process can be performed again at block 456.

If dry, at block 464, the inlet and outlet lines can be removed from the pack. A tag can be affixed to the pack with desired identifying information. The pack can optionally be enclosed in an outer container for storage. The method can end at block 470.

FIG. 5 illustrates a block diagram of an example machine 500 (e.g., computer system, computing device, machine, controller, etc.) that may be programmed into a special purpose machine suitable for implementing one or more embodiments for data processing, data communication, user interface, or like aspects disclosed herein with regards to the output device 240 and/or gas control system 120 or other automated controls of the systems and devices discussed above. For instance, the machine 500 may be embodied by or operably coupled to the output device 240 or gas control system 120, such as in the form of a computer or specialized electronic device that includes sufficient processing power, memory resources, and communications throughput capability to perform specific logic and functional operations for sensing, drying, and controlling nitrogen gas flow consistent with the examples herein.

The machine 500 may include a hardware processor 502 (e.g., a central processing unit (CPU), a graphics processing unit (GPU), a hardware processor core, or any combination thereof), a main memory 504 and a static memory 506, some or all of which may communicate with each other via an interconnect, link or bus 508. The machine 500 may further include a display unit 510, an alphanumeric input device 512 and a user interface (UI) navigation device 514. In an example, the display unit 510, alphanumeric input device 512 and navigation device 514 may be a touch screen display. The machine 500 may additionally include a storage device 516 (e.g., drive unit), a signal generation device 518 (e.g., an audio or radio signal generation device), and a network interface device 520 (e.g. for connectivity with a network). The machine 500 may include an output controller 528, such as a serial (e.g., universal serial bus (USB)), parallel, or other wired or wireless (e.g., infrared (IR), near field communication (NFC), etc.) connection to communicate or control one or more peripheral devices, and an input controller 530 to connect to more sensors.

The storage device 516 may include a machine readable medium 522 that is non-transitory on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the storage device 516 may constitute machine readable media.

The term “machine readable medium” may include any medium that is capable of storing, encoding, or carrying instructions for execution by the machine 500 and that cause the machine 500 to perform any one or more of the techniques of the present disclosure, or that is capable of storing, encoding or carrying data structures 527 used by or associated with such instructions. Non-limiting machine-readable medium examples may include solid-state memories, and optical and magnetic media. Specific examples of machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®), IEEE 802.15.4 family of standards, peer-to-peer (P2P) networks, among others. In an example, the network interface device 520 may include one or more physical jacks or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software.

Method examples or other operations described herein can be machine or device (e.g., computer, robotic) implemented at least in part. The components of the illustrative devices, systems and methods employed in accordance with the illustrated embodiments may be implemented, at least in part, in digital electronic circuitry, analog electronic circuitry, or in computer hardware, firmware, software, or in combinations of them. These components may be implemented, for example, as a computing program product such as a computing program, program code or computer instructions tangibly embodied in an information carrier, or in a machine-readable storage device, for execution by, or to control the operation of, a data processing apparatus such as a programmable processor, a computer, or multiple computers. A computing program may be written in any form of programming language, including compiled or interpreted languages, and it may be deployed in any form, including as a stand-alone program or as a module, component, subroutine, or other unit suitable for use in a computing environment. Also, functional programs, codes, and code segments for accomplishing the techniques described herein may be easily construed as within the scope of the present disclosure by programmers skilled in the art. Method steps associated with the illustrative embodiments may be performed by one or more programmable processors executing a computing program, code or instructions to perform functions (e.g., by operating on input data and/or generating an output). Method steps may also be performed by, and apparatus may be implemented as, special purpose logic circuitry, e.g., an FPGA (field programmable gate array) or an ASIC (application-specific integrated circuit), for example.

Thus, in implementation in a controller or other machine for medical item processing, various logical blocks, modules, and circuits described in connection with the embodiments disclosed herein may be implemented or performed with a general-purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Processors suitable for the execution of a computing program include, by way of example, both general and special purpose microprocessors, and any one or more processors of any kind of digital computer. Information carriers suitable for embodying computing program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, etc.). The processor and the memory may be supplemented by or incorporated in special purpose logic circuitry.

As used herein, “machine-readable medium” or “machine-readable storage medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” or “machine-readable storage medium” should be taken to include a single medium or multiple media (e.g., a centralized or distributed database, or associated caches and servers) able to store processor instructions. The term “machine-readable medium” or “machine-readable storage medium” shall also be taken to include any medium, or combination of multiple media, which is capable of storing instructions for execution by one or more processors (or other processing circuitry), such that the instructions, when executed by one or more processors cause the one or more processors to perform any one or more of the methodologies described herein. Accordingly, a “machine-readable medium” or “machine-readable storage medium” refers to a single storage apparatus or device, as well as “cloud-based” storage systems or storage networks that include multiple storage apparatus or devices. A non-transitory “machine-readable medium” or “machine-readable storage medium” as used herein excludes signals per se.

### Various Notes & Examples

In some aspects, the techniques described herein relate to a system including: a nitrogen source to provide nitrogen gas; a drying pack fluidly connected to the nitrogen source, the drying pack including: a hermetically sealable pouch shaped to receive an endoscope; an inlet block having one or more endoscope connections, the inlet block fluidly connected to the nitrogen source; an outlet block configured to exhaust the hermetically sealable pouch; a gas control system coupled to the drying pack, the control module adapted to adjust flow of the nitrogen gas into the drying pack.

In some aspects, the techniques described herein relate to a system, wherein the one or more endoscope connections provide respective fluid connections to channels of the endoscope, including: an air/water channel, a first suction channel, a second suction channel, and an auxiliary channel, or combinations thereof.

In some aspects, the techniques described herein relate to a system, wherein the inlet block is heat bonded to the hermetically sealable pouch.

In some aspects, the techniques described herein relate to a system, wherein the outlet block is heat bonded to the hermetically sealable pouch.

In some aspects, the techniques described herein relate to a system, wherein the pouch is sealable along one edge of the pouch with a resealable press-to-close zipper or a resealable clamp.

In some aspects, the techniques described herein relate to a system, wherein the gas control system includes a plurality of valves to regulate flow of nitrogen in the system.

In some aspects, the techniques described herein relate to a system, wherein the gas control system includes a nitrogen gas pressure-reducing regulator with one or more gauges, a manually or automatically actuated flow control needle valve, and a mass flow meter.

In some aspects, the techniques described herein relate to a system, wherein the gas control system comprises a pressure relief valve and a pressure sensor to control pressure of the nitrogen gas introduced into the drying pack.

In some aspects, the techniques described herein relate to a system, further including a user device configured to provide a graphical user interface, the user device in communication with the gas control system.

In some aspects, the techniques described herein relate to a system, wherein the nitrogen source is configured to provide ultra-high purity nitrogen gas of at least grade N5.3.

In some aspects, the techniques described herein relate to a system, wherein the gas control system includes a temperature regulation element.

In some aspects, the techniques described herein relate to a pack, wherein the temperature regulation element is configured to maintain the temperature of the drying pack from about 45° C. to about 55° C.

In some aspects, the techniques described herein relate to a pack, wherein the temperature regulation element is a resistance coil or an infrared heater.

In some aspects, the techniques described herein relate to a drying pack for drying an endoscope, the pack including: a pouch sized to accommodate a coiled endoscope on a support tray provided therein, wherein the pouch is subsequently sealable along one side; an endoscope connection manifold bonded to an interior of the pouch, wherein the manifold is actuatable to fluidly connect respective hook-up lines to one or more internal channels of the endoscope; an inlet valve configured to receive a fluid, the inlet valve fluidly connected to the respective hook-up lines via the manifold; and an exhaust valve fluidly connectable to the interior of the pouch, wherein, when the pack is connected to a fluid source, the fluid is configured to enter the pouch through the inlet valve, flow through the manifold and the respective hook-up lines, flow through the one or more internal channels of the endoscope, exit the endoscope at the distal tip of the endoscope, flow inside the pouch around the pouch in a circulatory manner around the outside of the endoscope, and exit the pouch through the exhaust valve.

In some aspects, the techniques described herein relate to a pack, wherein the fluid is ultra-high purity nitrogen gas of at least grade N5.3.

In some aspects, the techniques described herein relate to a pack, further including a baffle located inside the pouch, the baffle shaped to allow for winding of the endoscope therearound.

In some aspects, the techniques described herein relate to a pack, wherein the fluid is configured to flow in a cyclonic flow within the interior of the pouch.

In some aspects, the techniques described herein relate to a pack, wherein the pouch includes a double-track press-to-close zipper or a resealable gasketed clamp along one side of the pouch to seal the pouch.

In some aspects, the techniques described herein relate to a pack, further including an incoming fluid line connectable to the inlet valve.

In some aspects, the techniques described herein relate to a pack, wherein the inlet valve is configured to automatically close when the incoming fluid line is disconnected from the fluid source.

In some aspects, the techniques described herein relate to a pack, wherein the fluid source is controlled by a gas control system having a flow control needle valve that is manually or automatically actuated to control a flow rate of the fluid to the pouch.

In some aspects, the techniques described herein relate to a pack, wherein the fluid source is further controlled by a gas control system having a pressure sensor and a pressure relief valve that is manually or automatically actuated to control pressure of the fluid to the pouch.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system comprising:
a nitrogen source to provide nitrogen gas;
a drying pack fluidly connected to the nitrogen source, the drying pack comprising:
a hermetically sealable pouch shaped to receive an endoscope;
an inlet block having one or more endoscope connections, the inlet block fluidly connected to the nitrogen source; and
an outlet block configured to exhaust the hermetically sealable pouch; and
a gas control system coupled to the drying pack, the gas control system adapted to adjust flow of the nitrogen gas into the drying pack.

2. The system of claim 1, wherein the one or more endoscope connections provide respective fluid connections to channels of the endoscope, including: an air/water channel, a first suction channel, a second suction channel, and an auxiliary channel, or combinations thereof.

3. The system of claim 1, wherein the inlet block is heat bonded to the hermetically sealable pouch.

4. The system of claim 1, wherein the outlet block is heat bonded to the hermetically sealable pouch.

5. The system of claim 1, wherein the pouch is sealable along one edge of the pouch with a resealable press-to-close zipper or a resealable clamp.

6. The system of claim 1, wherein the gas control system comprises a plurality of valves to regulate flow of nitrogen in the system.

7. The system of claim 1, wherein the gas control system comprises a nitrogen gas pressure-reducing regulator with one or more gauges, a manually or automatically actuated flow control needle valve, and a mass flow meter.

8. The system of claim 7, wherein the gas control system comprises a pressure relief valve and a pressure sensor to control pressure of the nitrogen gas introduced into the drying pack.

9. The system of claim 1, further comprising a user device configured to provide a graphical user interface, the user device in communication with the gas control system.

10. The system of claim 1, wherein the nitrogen source is configured to provide ultra-high purity nitrogen gas of at least grade N5.3.

11. The system of claim 1, wherein the gas control system comprises a temperature regulation element.

12. The system of claim 11, wherein the temperature regulation element is configured to maintain the temperature of the drying pack from about 45° C. to about 55° C., and wherein the temperature regulation element is a resistance coil or an infrared heater.

13. A drying pack for drying an endoscope, the pack comprising:
a pouch sized to accommodate a coiled endoscope on a support tray provided therein, the pouch sealable along one side;
an endoscope connection manifold bonded to an interior of the pouch, the manifold actuatable to fluidly connect respective hook-up lines to one or more internal channels of the endoscope;
an inlet valve configured to receive a fluid, the inlet valve fluidly connected to the respective hook-up lines via the manifold; and
an exhaust valve fluidly connectable to the interior of the pouch,
wherein, when the pouch is connected to a fluid source, the fluid is configured to enter the pouch through the inlet valve, flow through the manifold and the respective hook-up lines, flow through the one or more internal channels of the endoscope, exit the endoscope at a distal tip of the endoscope, flow inside the pouch around an exterior of the endoscope, and exit the pouch through the exhaust valve.

14. The pack of claim 13, wherein the fluid is ultra-high purity nitrogen gas of at least grade N5.3.

15. The pack of claim 13, further comprising a baffle located inside the pouch, the baffle shaped to allow for winding of the endoscope therearound, and wherein the fluid is configured to flow in a cyclonic flow within the interior of the pouch.

16. The pack of claim 13, wherein the pouch includes a double-track press-to-close zipper or a resealable gasketed clamp along one side of the pouch to seal the pouch.

17. The pack of claim 13, further comprising an incoming fluid line connectable to the inlet valve.

18. The pack of claim 17, wherein the inlet valve is configured to automatically close when the incoming fluid line is disconnected from the fluid source.

19. The pack of claim 17, wherein the fluid source is controlled by a gas control system having a flow control needle valve that is manually or automatically actuated to control flow rate of the fluid to the pouch.

20. The pack of claim 19, wherein the fluid source is further controlled by the gas control system having a pressure sensor and a pressure relief valve that is manually or automatically actuated to control pressure of the fluid to the pouch.
